(19) United States
(12) Patent Application Publication
(10) Pub. No.: US 2020/0359928 A1
(43) Pub. Date: Nov. 19, 2020

(54) NOISE PREVENTING EEG RECORDING CAP

(71) Applicant: DOKUZ EYLUL UNIVERSITESI REKTORLUGU, Izmir (TR)

(72) Inventor: Hikmet GÜMÜS, Izmir (TR)

(21) Appl. No.: 16/957,989

(22) PCT Filed: Dec. 10, 2018
(86) PCT No.: PCT/TR2018/050782

§ 371 Date: Jun. 25, 2020

(30) Foreign Application Priority Data
Dec. 29, 2017 (TR) 2017/22875

(51) Int. Cl. A61B 5/0476; A61B 5/00

(52) U.S. Cl. CPC A61B 5/0476; A61B 5/6803; A61B 2560/0214; A61B 2562/182; A61B 2562/14; A61B 5/7203

(57) ABSTRACT

A noise preventing EEG recording cap that reduces the noise that occurs during EEG recording. The EEG cap mainly includes an inner layer that separates the electronic recording section, Electromagnetic isolation middle layer, the outer area of the cap that provides water impermeability, a fluid transfer channel onto which hydrogel is applied for situations besides under water usage, an ADC unit provided for each electrode located in the signal layer, DC power supply that prevents the noise caused by the electric line, a secondary recording unit that enables use of different algorithms in order to prevent artifacts and to obtain other noise sources, that allows other electrophysiological noises and artifact sources to be simultaneously recorded and as a result allows these sample in time intervals where noise is not present and the records to be separated, and a DSP that processes signals on the data recording unit.

### TECHNICAL FIELD OF THE INVENTION

The invention subject to the application is related to a cap which reduces the noise that can occur and which can be used during EEG recording.

### Known State of the Art (Prior Art)

The recording of electrical activities of the brain is a difficult and complex process. During this process a cap which is placed on the skull of the person for recording and electrodes that are in contact with the scalp is used. The currents used during these measurements carried out on the scalp is around 10-100 μV. In order for such low values to be able to be recorded and evaluated, these currents are passed through different bioinstrumentation systems and are increased and recorded. However this situation leads to increasing noise and artefacts besides the information received from the source.

In order to prevent this obtained signals are filtered. However in this case, original signals are also deformed together with the artefacts that are desired to be cleaned.

It is possible to classify EEG artefacts physiologically and extra-physiologically. The physiological artefacts can be listed as EMG (electrical signals that increase the amplitude received from the muscles), ECG (signals that show the electrical activity of the heart), Glossokinetic (signals received from the tongue and related area), Eye movements (electrical signals received from the eye and the muscles surrounding the eye), artefacts that are formed due to respiration, and artefacts that are caused by sweating and ion changes on the surface skin from where the measurements are being taken.

The non physiological artefacts can be listed as artefacts arising from electrode impedance, artefact that can vary from area to area arising from the electrical connection used (50 hz, 60 hz), artefacts caused by other living beings in the medium, electromagnetic noise sources in the medium (all electronic devices and systems used for recording is included).

They can differ according to the measurements carried out but they can be around 128 or even 256. In this case, due to the number of cables protruding out of the CAP, the ability to move is reduced and as there are too many cables and they are close to each other, this may impair the quality of the signals.

Due to the structure of the Cap and the artefacts that may be caused due to the motion that is formed, EEG measurements are usually taken in sleep or when the patient is motionless. In conditions where the patient is moving, particularly during exercise, the artefacts that are formed in the records increases.

As the electrodes are exposed, this prevents recording outdoors or in environments such as the sea, etc.

The invention has been developed in order to eliminate the disadvantages mentioned above and is related to a noise preventing EEG recording cap.

### Brief Description of the Invention and its Aims

The present invention has been developed in order to eliminate the disadvantages mentioned above and is related to a noise preventing EEG recording cap in order to provide new advantages to the related technical field.

The invention comprises an EEG CAP and a data recording medium developed in order to reduce artefacts occurring during an EEG record.

The aim of the invention is to provide a system which can perform EEG recording under water.

Another aim of the invention is to provide a system that can increase the signal quality of EEG recording in non static environments.

Another aim of the invention is to provide a system which enables mobile usage having high signal quality.

Another aim of the invention is to provide a system which enables recording in mediums where it is believed that EEG recording is not possible such as factories, vehicles, and machinery in and around which intensive electromagnetic pollution is present.

According to an embodiment of the invention the CAP has a secondary recording unit that is positioned outside the cap.

According to an embodiment the invention comprises an ADC unit which is placed on the electrode and which does not have a cable connection line between the electrode and itself.

According to an embodiment the invention comprises an ADC unit where digitalization is carried out when the signal is still at the electrode.

According to another embodiment of the invention, the invention comprises a liquid transfer channel located on the external layer which enables external fluids that has been formed due to sweating or other similar reasons, to be removed.

According to another preferred embodiment of the invention, the invention comprises a DC power supply (battery) placed on the CAP.

According to another preferred embodiment, the invention comprises additional signal inputs which enables to receive signals from the closest area to the primary noise source and removes this noise from the main signal.

According to another preferred embodiment of the invention, the invention comprises an outer layer formed of latex or silicon material.

According to another preferred embodiment of the invention, the invention comprises an outer layer formed of flexible polymer material.

According to another preferred embodiment of the invention, the invention comprises an outer layer which does not have cables thereon that can be observed from the outside.

According to another preferred embodiment of the invention, the invention comprises an electromagnetic isolation web that is placed between the middle layer and the inner layer.

### Definitions of the Parts/Aspects/Sections Forming the Invention

The parts/sections/aspects provided in the figures that have been prepared in order to further describe the noise preventing EEG recording cap developed by means of the invention have each been numbered and the references of each number has been listed below.
1 Inner layer
2 Middle layer
3 Outer layer
4 Liquid transfer channel
5 Isolation web
6 ADC unit
7 electrode
8 DC power supply
9 band section
10 secondary recording unit

### DEFINITION OF THE FIGURES OF THE INVENTION

The figures that have been prepared in order to further describe the noise preventing EEG recording cap developed by means of the invention has been described below.

FIG. 1, is the general view of the EEG Cap.

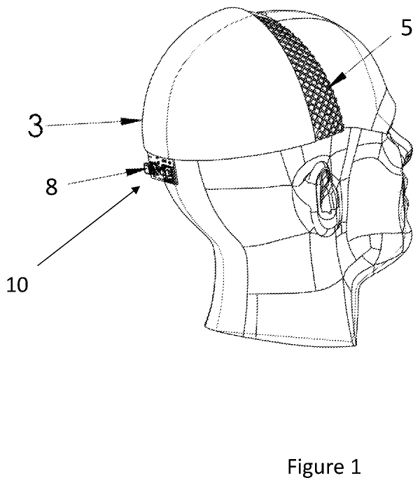

FIG. 2, is the section view of the EEG Cap.

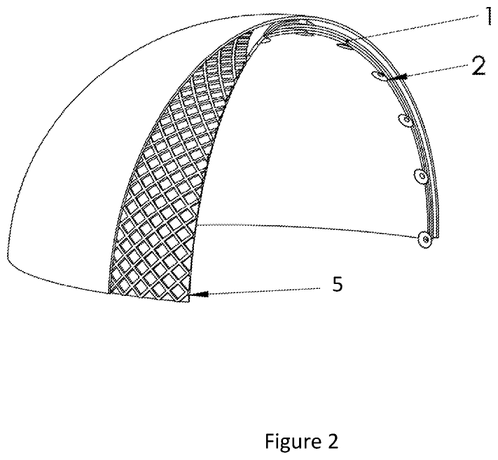

FIG. 3, is the side view of the impermeability section of the EEG Cap.

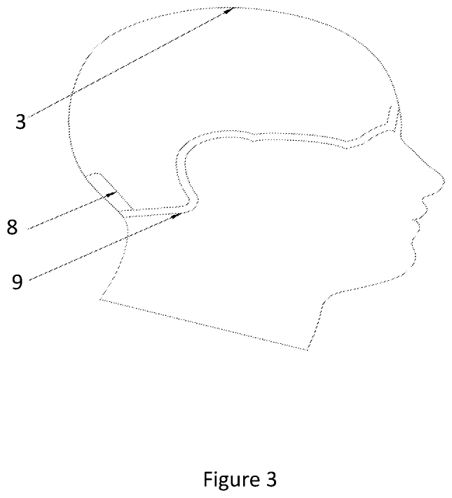

FIG. 4, is the view of the EEG Cap layers.

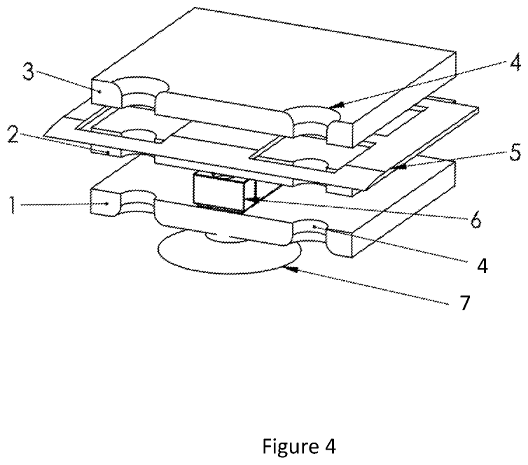

### DETAILED DESCRIPTION OF THE INVENTION

The novelty of the invention has been described with examples that shall not limit the scope of the invention and which have been intended to only clarify the subject matter of the invention. The EEG CAP and a data recording medium that has been developed in order to reduce artefacts occurring during an EEG record has been described in detail below.

By means of the cap formed of latex or other similar flexible polymers, electrodes (7) are enabled to be fixed on the scalp even though the users may have different sized head structures, and it is ensured that the electrodes (7) do not move out of position even if the user is moving.

The metal web layer which is located on the signal layer prevents external electromagnetic effects and increases signal quality.

The amount of flexibility of the coils used in the metal web according to the structure and transition features thereof is determined and caps having different flexibility levels according to different usage fields are formed.

The intensity of the metal web shall change according to the signal features and can be developed in order to prevent different wave lengths.

Data shall be digitalized at the electrode level with ADC located in the signal layer and the ADC shall also ensure the data to be protected. As a result the metal web and the cabling formed following ADC will not be affected from motion and external electromagnetic effects.

As data is digitalized, it can be recorded into the recording unit located inside the signal layer and as a result all of the recording process is carried out inside the metal web and therefore the artefacts that may be formed will be reduced. As there is no cable or similar medium that shall protrude out of the car during recording, any effects that may limit motion will not be present.

In the case that data is transferred outside of the Cap with a cable, and as data is digitalized, it can be transferred with a single coil, shielded cable and motion restrictions and similar effects shall be minimized.

The impermeability section having different density forming the borders of the CAP, generally shall restrict the motion of the CAP and shall allow recording under water.

The secondary recording unit and the artefact source outside the CAP, allows other electrophysiological noise to be simultaneously recorded and as a result these records allows sample in time intervals where noise is not present and the records to be separated. The recording of other noise sources shall allow the usage of different algorithms in order to prevent artefacts and perform filtering.

The DC power supply located on the CAP prevents the 50-60 hz noise caused by the electric line.

The factors that may affect the quality of recording are eliminated by means of the thermal detection and sweating sensor located inside the cap.

The hydro channels located on the surface of the cap, helps to discharge sweat and reduces the ion changes that may be caused by sweating during recording and therefore reduces noise. The hydrophilic gel that shall be applied on these channels shall enable the fluid and sweat formed on said surface to be drawn out and discharged.

The EEG cap has several different layers. EEG cap layers are formed of: An inner layer, middle layer, outer layer, optional sweat, fluid transfer channel, electromagnetic isolation web, ADC unit, electrode and second optional sweat, fluid transfer channel. Moreover the invention comprises additional signal inputs which enables to receive signals from the closest area to the primary noise source (eyes, chin etc) and removes this noise from the main signal.

The electrode (7) and the adc unit (6) are located inside two layers as a single unit that is physically integrated with each other.

If we look into these layers, we can say that these layers are as follows;
An inner layer (1) which separates the electronic recording section,
Electromagnetic isolation layer middle layer (2),
The outer layer (3) which is the outer area of the CAP which provides water impermeability,
The optional sweat, fluid transfer channel (4) onto which hydro gel shall be applied for situations besides under water usage, which receives the fluid from the electrodes and transfers it to the hydrophilic section,
Electromagnetic isolation web (5), which provides electromagnetic isolation and which is the web section,
An ADC unit (6) provided at least one for each electrode (7) or electrode (7) group located in the signal layer,
A channel which receives fluid from the electrodes and transfers it to the hydrophilic section which is an optional sweat, fluid transfer channel (4) for situations besides under water usage, and
Electrode

Noise preventing EEG recording cap characterized in that it comprises;
An inner layer (1) which separates the electronic recording section,
Electromagnetic isolation layer middle layer (2),
Outer layer (3) which is the outer area of the CAP which provides water impermeability,
At least an optional sweat, fluid transfer channel (4) onto which hydro gel shall be applied for situations besides under water usage, which receives the fluid from the electrodes and transfers it to the hydrophilic section,
Electromagnetic isolation web (5), which provides electromagnetic isolation and which is the web section,
An ADC unit (6) provided at least one for each electrode (7) or electrode (7) group located in the signal layer,
An electrode (7) which receives the signals,
Signal inlets into which the electrodes (7) are fixed,
DC power supply (battery) (8) which prevents the 50-60 hz noise caused by the electric line.
A secondary recording unit (10) which enables to use different algorithms in order to filter and prevent artefacts and to obtain other noise sources, which allows other electrophysiological noises and artefact sources to be simultaneously recorded and as a result allows these sample in time intervals where noise is not present and the records to be separated,
Band section (9) which provides impermeability,
And a DSP (Digital Signal Processor) which allows to process signals on the data recording unit.

1. A noise preventing EEG recording cap, comprising:
an inner layer which separates an electronic recording section;
an electromagnetic isolation layer middle layer;
an outer layer which is the outer area of the cap which provides water impermeability;
at least an optional sweat, fluid transfer channel onto which hydro gel shall be applied for situations besides under water usage, which receives fluid from the electrodes and transfers it to the hydrophilic section,
an electromagnetic isolation web which provides electromagnetic isolation and which is the web section;
an ADC unit provided at least one for each electrode or electrode group located in the signal layer;
an electrode which receives the signals;
signal inlets into which the electrodes are fixed;
a DC power supply (battery) which prevents 50-60 hz noise caused by the electric line;
a secondary recording unit which enables to use different algorithms in order to filter and prevent artefacts and to obtain other noise sources, which allows other electrophysiological noises and artefact sources to be simultaneously recorded and as a result allows these sample in time intervals where noise is not present and the records to be separated;
a band section which provides impermeability; and
a DSP (Digital Signal Processor) which allows to process signals on the data recording unit.

2. A noise preventing EEG recording cap according to claim 1, comprising a secondary recording unit which is positioned outside the CAP.

3. A noise preventing EEG recording cap according to claim 1, comprising an ADC unit which is placed on the electrode and which does not have a cable connection line between the electrode and itself.

4. A noise preventing EEG recording cap according to claim 1, comprising a liquid transfer channel located on the external layer which enables external fluids that have been formed due to sweating or other similar reasons, to be removed.

5. A noise preventing EEG recording cap according to claim 1, comprising a DC power supply (battery) positioned on the CAP.

6. A noise preventing EEG recording cap according to claim 1 comprising additional signal inputs which enables to receive signals from the closest area to the primary noise source and removes this noise from the main signal.

7. A noise preventing EEG recording cap according to claim 1, comprising an outer layer formed of latex material.

8. A noise preventing EEG recording cap according to claim 1, comprising an outer layer (3) formed of flexible polymer material.

9. A noise preventing EEG recording cap according to claim 1, comprising an outer layer (3) formed of silicon material.

10. A noise preventing EEG recording cap according to claim 1, comprising a structure which makes it easier for the electrodes to be fixed on the scalp even though the users may have different sized head structures, and ensures that said electrodes do not move out of position even if the user is moving.

11. A noise preventing EEG recording cap according to claim 1, having an electromagnetic isolation web comprising a helical structure.

12. A noise preventing EEG recording cap according to claim 1, comprising an electromagnetic isolation web where the depth of the metal web is changed according to the signal features to be received and can be changed in order to prevent different wave lengths.

13. A noise preventing EEG recording cap according to claim 1, comprising an outer layer which does not have cables that are exposed and can be observed from outside.

14. A noise preventing EEG recording cap according to claim 1, comprising an electromagnetic isolation web that is placed between the middle layer and the outer layer.

15. A noise preventing EEG recording cap according to claim 1, comprising electrodes that are positions under the inner layer.
